(19) United States
(12) Patent Application Publication
(10) Pub. No.: US 2025/0359859 A1
(43) Pub. Date: Nov. 27, 2025

(54) LIQUID DISPENSER WITH CONTROLLED DISPENSING

(71) Applicant: Solventum Intellectual Properties Company, Maplewood, MN (US)

(72) Inventors: Dominic Nolan, Salisbury (GB); Alan R. Dombrowski, Woodbury, MN (US); Joseph Dartnell, Poole (GB); Val Vossen, Minneapolis, MN (US); Benjamin A. Pratt, Poole (GB); John Cain, River Falls, WI (US)

(21) Appl. No.: 18/872,332

(22) PCT Filed: Jun. 6, 2023
(86) PCT No.: PCT/IB2023/055812

§ 371 Date: Dec. 6, 2024

Related U.S. Application Data
(60) Provisional application No. 63/350,510, filed on Jun. 9, 2022.

(51) Int. Cl. A61B 17/00

(52) U.S. Cl. CPC A61B 17/00491; A61B 2017/00367; A61B 2017/00495

(57) ABSTRACT

The disclosed dispenser provides for controlled dispensing of a liquid. The dispenser has a housing that supports a fluid holding system, fluid delivery system, priming mechanism, and an actuation control mechanism. The priming mechanism creates and stores an actuation force, which breaks the sealed fluid delivery system and the force can be controllably released with an actuation control mechanism to dispense the liquid. The actuation control mechanism includes a plunger that slidably advances into fluid-containing cylinders from force by the priming mechanism. The actuation control mechanism further includes an actuation button that interlocks with the plunger to start and stop dispensing. Optionally, the dispenser has two cylinders and a mixing chamber for dispensing a multi-component liquid, such as an adhesive.

### TECHNICAL FIELD

The present disclosure relates to a liquid dispenser with controlled dispensing of the liquid. In particular, the liquid dispenser includes a priming mechanism for creating and storing an actuation force, which can be controllably released with an actuation control mechanism to dispense the liquid.

### BACKGROUND

Staples and sutures are commonly used for wound closure in operating room settings. While they are used for high tension and large closures, tissue adhesives alone or in combination with inner deep sutures are also used for small wounds. Tissue adhesives can be less painful, can lead to a better cosmetic outcome and provide an antimicrobial barrier against further infections. As compared to staples and sutures, tissue adhesives can also be applied rapidly. Common tissue adhesives are cyanoacrylate-based compositions which have very low viscosities. In addition, the dispensers are often a sealed tube containing the tissue adhesive, where once the opening is unsealed, the tube is simply squeezed to deliver the low viscosity adhesive. Therefore, these commonly used tissue adhesives are not easily controlled upon delivery to a wound.

### SUMMARY

The disclosed dispenser provides for controlled dispensing of a liquid. The dispenser has a housing that supports a fluid holding system, fluid delivery system, priming mechanism, and an actuation control mechanism. The priming mechanism creates and stores an actuation force, which breaks the sealed fluid delivery system and the force can be controllably release with an actuation control mechanism to dispense the liquid. The actuation control mechanism includes a plunger that slidably advances into fluid-containing cylinders from force by the priming mechanism. The actuation control mechanism further includes an actuation button that interlocks with the plunger to start and stop dispensing. Optionally, the dispenser has two cylinders and a mixing chamber for dispensing a multi-component liquid.

Priming the priming mechanism by moving from a first unprimed position to a second primed position, compresses the spring and also translates the fluid holding system into engagement with the fluid delivery system. In particular, the sealed container holding the fluid is pierces and the contained fluid then is in communication with the fluid delivery system. The energy stored in the spring from the priming is then controllably released via the actuation control mechanism.

In one embodiment, the liquid dispenser has a proximal end and a distal end, and a housing the supports the fluid holding system, a fluid delivery system, a priming mechanism, and an actuation control mechanism with an applicator tip at the distal end. The fluid holding system comprises a first cylinder with a seal for holding a first liquid, a first piston slidably within the first cylinder. The fluid delivery system comprises a first piercing element and a nozzle extending from the first cylinder to the applicator tip. The priming mechanism comprises a priming button slidably connected to the housing, a spring seated within the priming button, a priming latch extending from the priming button. The actuation control mechanism comprises a plunger slidably connected to the housing comprising a first fluid dispensing arm engaging with the first piston, a plurality of plunger engagement extensions. Each plunger engagement extension is spaced from the adjacent plunger engagement extension. A spring abutment surface contacting the spring. An actuation button connected with a plurality of actuation engagement extensions. Each actuation extension is spaced from the adjacent actuation extension by an actuation extension gap. The priming button is movable from a first unprimed position to a second primed position where in the second primed position (i) the priming latch locks with a priming active retainer in the housing to restrict movement in the proximal direction and to compress the spring against the spring abutment surface of the plunger (ii) and engage the piercing element though the seal into the first cylinder.

In one embodiment, the actuation button is movable from a first engaged position with the actuation engagement extensions interconnected with the plunger engagement extensions to restrict movement of the plunger to a second disengaged position with the actuation engagement extension disengaged from the plunger engagement extensions.

In one embodiment, the fluid holding system further comprises a second cylinder holding a second liquid and a second piston slidably within the second cylinder, and wherein the plunger further comprises a second fluid dispensing arm engaging with the second piston.

The actuation button is in the first engaged position in a resting position, and displacement of the actuation button into the housing moves to the second disengaged position. The priming button in the first unprimed position the priming latch locks with a priming rest retainer in the housing to restrict movement in the proximal direction.

In one embodiment, the method of dispensing liquid from the dispenser comprises moving the priming mechanism to the second primed position, wherein the priming latch is retained in the priming active retainer and the spring is compressed against the spring abutment surface and actuating the actuation button to disengage the actuation engagement extensions from the plunger engagement extensions such that the spring slidably moves the spring abutment surface of the plunger and the first fluid displacement arm in the first fluid cylinder. Further, the method comprises actuating the actuation button to engage the actuation engagement extensions the plunger engagement extensions to stop slidable movement of the plunger.

### BRIEF DESCRIPTION OF DRAWINGS

FIG. 1 is a section view of one embodiment of a liquid dispenser with controlled dispensing;

FIG. 2 is an exploded perspective view of the disassembled dispenser of FIG. 1;

FIG. 3 is a section view of the assembled dispenser of FIG. 1 in a stored, un-primed position;

FIG. 4 is a section view of the assembled dispenser of FIG. 1 in a primed position and with the actuation control mechanism unreleased and not dispensing liquid;

FIG. 5 is a section view of the assembled dispenser of FIG. 1 in a primed position and with the actuation control mechanism partially released and dispensing liquid;

FIG. 6 is a section view of the assembled dispenser of FIG. 1 in a primed position and with the actuation control mechanism fully released and having fully dispensed the liquid;

FIG. 7 is a perspective, section view of the actuation control mechanism of the liquid dispenser of FIG. 3;

FIG. 8 is a section view of the assembled dispenser of FIG. 4 showing the path of liquid flow from the dispenser;

FIG. 9A is a perspective view of one embodiment of an applicator tip for the liquid dispenser;

FIG. 9B is a front, perspective view of an embodiment of an applicator tip for the liquid dispenser;

FIG. 9C is a perspective view of an embodiment of an applicator tip for the liquid dispenser;

FIG. 9D is a side sectional view of an applicator tip for the liquid dispenser;

FIG. 10A-10F are perspective views of embodiments of a piercing element;

FIG. 11 is a perspective view of a second embodiment of the liquid dispenser.

While the above-identified drawings and figures set forth embodiments of the invention, other embodiments are also contemplated, as noted in the discussion. In all cases, this disclosure presents the invention by way of representation and not limitation. Numerous other modifications and embodiments can be devised by those skilled in the art, which fall within the scope and spirit of this invention. The figures may not be drawn to scale.

### DETAILED DESCRIPTION

Various designs for a liquid dispenser are disclosed. The disclosed liquid dispenser 100 includes a priming mechanism 400 that creates and stores an actuation force, which can be controllably released with an actuation control mechanism 500 to dispense the contained liquid 311.

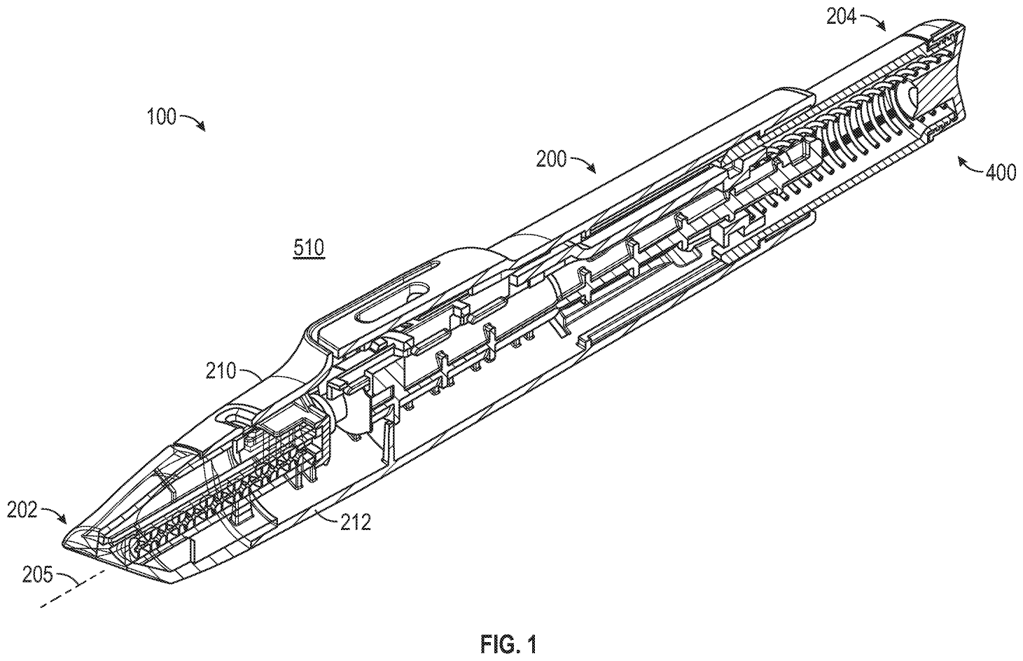

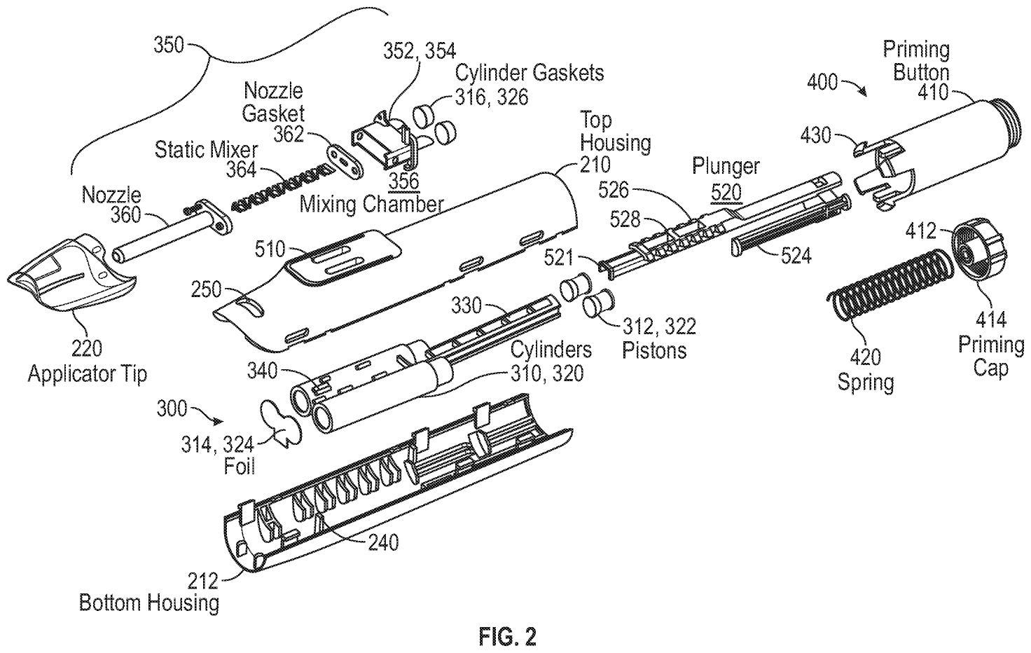

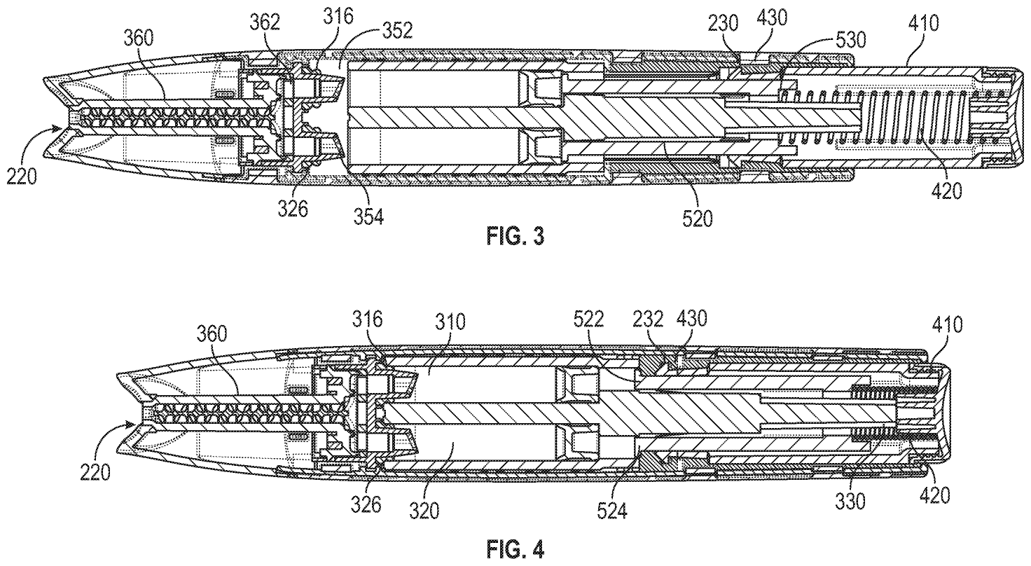

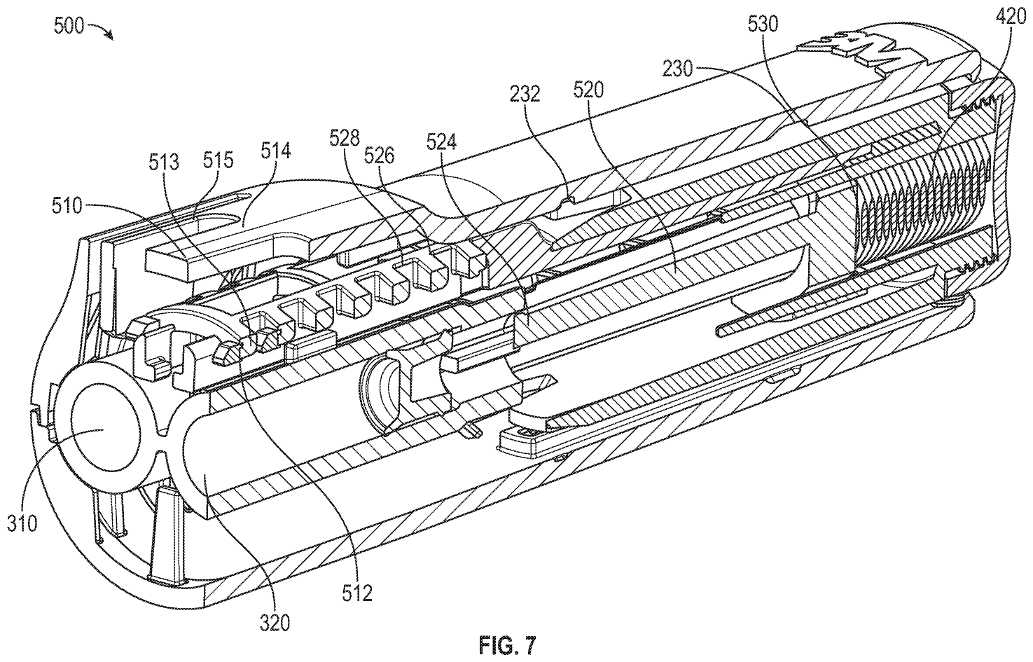

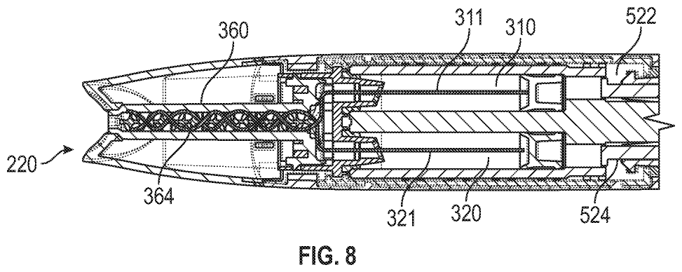

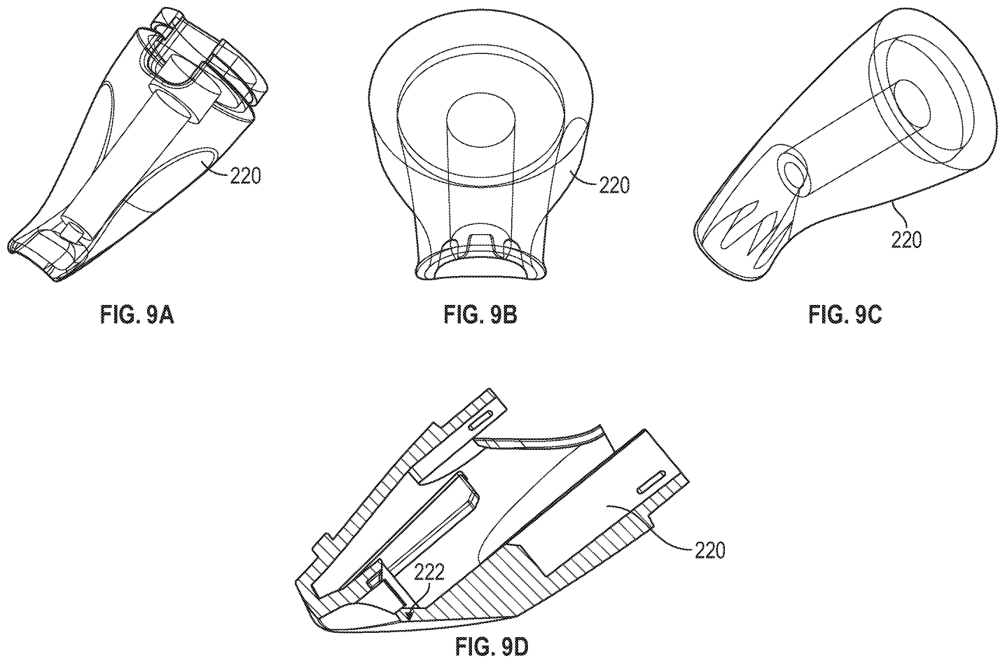

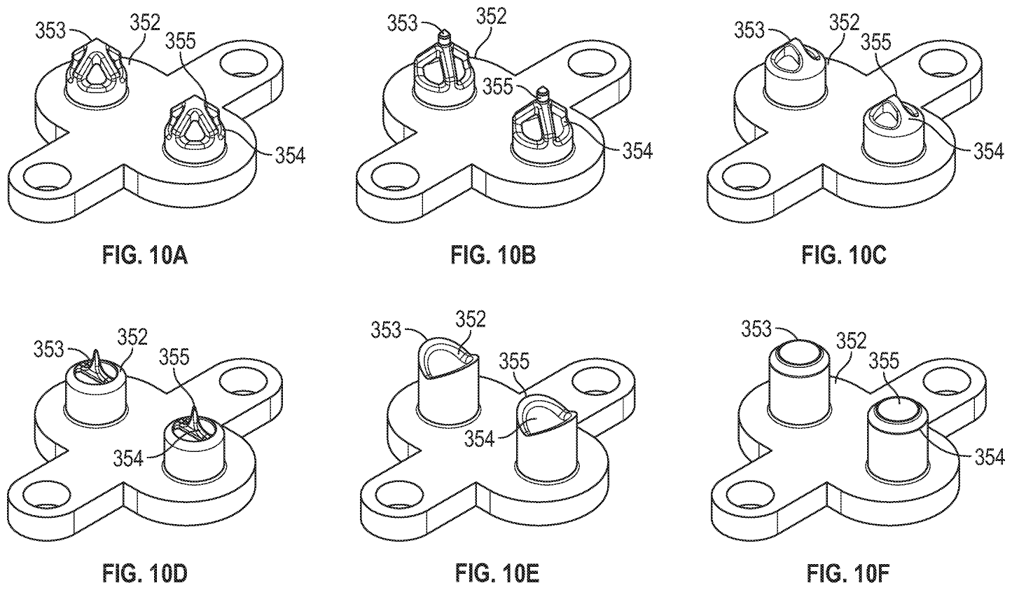

FIG. 1 is a perspective view of a liquid dispenser 100 for controlled dispensing. FIG. 2 is an exploded perspective view of the disassembled dispenser 100 of FIG. 1. FIGS. 3-6 show the actuation process of the dispenser 100. FIG. 7 shows the actuation control mechanism 500 of the dispenser 100. FIG. 8 shows a first liquid 311 and second liquid 321 dispensing from the dispenser 100. FIGS. 9A-9E show alternative embodiments of applicator tips 220. FIGS. 10A-10F show alternative embodiments of piercing elements 352, 354. Common reference numbers among the figures will be used for common elements.

The dispenser 100 includes a housing 200. The housing 200 supports, holds, or contains, some or all of the components. The dispenser 100 includes a fluid holding system 300, a fluid delivery system 350, a priming mechanism 400, and an actuation control mechanism 500, each of which will be described in more detail below. The housing 200 is fixed, while the some or all of the fluid holding system 300, priming mechanism 400, and actuation control mechanism 500 move relative to the housing 200

The elements of the dispenser 100 will be described, which is best seen in FIGS. 2, 7, 9A-9E, and 10A-10F. The relative movement of the dispenser 100 will be described in FIGS. 3-6.

### Housing

As seen in FIG. 1, the housing 200 has a distal end 202 and a proximal end 204, opposite from the distal end 202. Generally, the housing 200 extends along a longitudinal axis 205. An applicator tip 220 is at the distal end 202 from which the contained liquid 311 is dispensed. Typically, for ease of assembly, the housing 200 has a top housing 210 and a bottom housing 212 that connect together to contain the inner components. Various support features can be formed within the housing 100 to provide structural stability to the housing 100 itself, and to provide resting surfaces for the components contained within the housing 200

Within the wall of either the top housing 210 or bottom housing 212, or both, is a priming rest retainer 230 and a priming active retainer 232 (see FIG. 3-6). The priming rest retainer 230 receives the priming latch 430 of the priming button 410 when the priming button 410 is in the first unprimed position (FIG. 3). The priming active retainer 232 receives the priming latch 430 when the priming button is actuated to the second primed position (FIG. 4). In one embodiment, the dispenser 100 is a single use item and so for both the priming rest retainer 230 and priming active retainer 232, the interface with the priming latch 430 is such that the priming mechanism 400 can slide from the proximal end 204 to the distal end 202, without releasing back in the proximal end 204. In other embodiments, the dispenser 100 could be reusable, and therefore, the priming latch 430 could be released via actuation from either the priming rest retainer 230 or the priming active retainer 232 such that the priming mechanism 400 can be released and reactivated.

Within the housing 200, extending from either the top housing 210 or bottom housing 212, or both, is a cylinder retainer 240. The cylinder retainer 240 is a deformable projection that holds the fluid holding system 300 in a position prior to activation by the priming mechanism 400. This can provide stability during storage and prevent damage or leaks. Upon activation of the priming mechanism 400, sufficient force is applied to deform, break, or collapse the cylinder retainer 240 and allow for the fluid holding system 300 to translate within the housing 200 in a direction toward the distal end 202, as shown in FIG. 4. This translation allows the cylinders to be pierced by the piercing mechanism of the fluid delivery system.

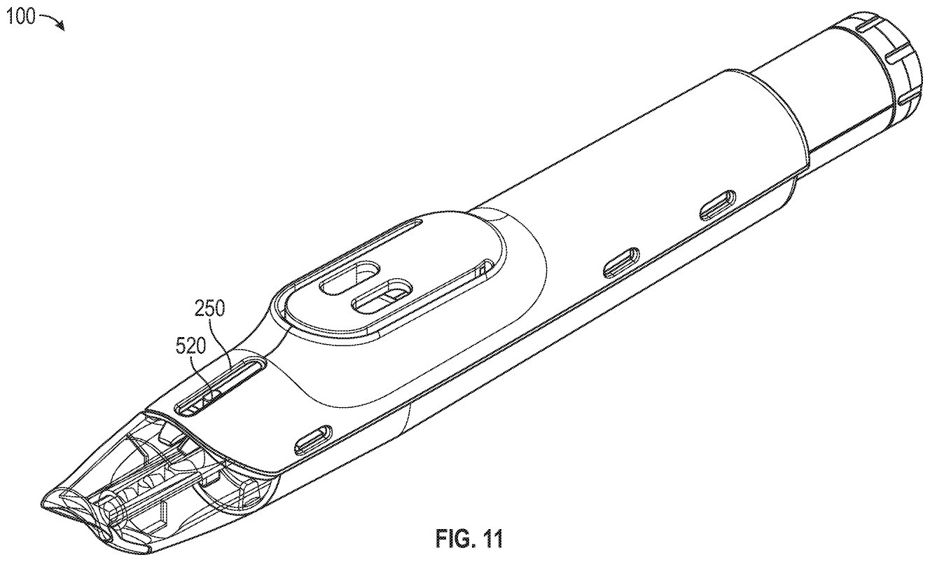

The housing 200 can include a delivery indicator opening 250 such as shown in FIG. 2 or FIG. 11. As will be described in more detail below, the actuation control mechanism 500 slidably moved during dispensing, and a portion of the actuation control mechanism 500 can be apparent though this delivery indicator opening 250. This can provide a visual indication to the user when the how much of the remining dispensing distance remains, giving an indication of how much liquid remains to be dispensed.

### Fluid Holding System

As best seen in FIGS. 1 and 2, the fluid holding system 300 has a first fluid cylinder 310, a first piston 312 at the proximal end 204, and a first seal 314. The first fluid cylinder 310 hold the first liquid 311. (FIG. 8) The first piston 312 fits within the first fluid cylinder 310 and is slidable within the first cylinder 310. The first liquid 311 can be any number of viscous components. For example, the first liquid 311 could be an adhesive, a skin protectant, oil, or solvent.

As shown in FIGS. 2 and 3, optionally, the fluid holding system 200 further has a second fluid cylinder 320 holding a second liquid 321. The second fluid cylinder 320 also has a second piston 322 that fits within the second fluid cylinder 320.

Typically, during storage and prior to use, the first liquid 311 is sealed in the first fluid cylinder 310 by a first seal 314, and the second liquid 321 is sealed within the second fluid cylinder 320 by a second seal 324. The seal 314, 324 is typically a puncturable film and may be a multilayer laminate that is welded or otherwise secured to the cylinders to for a robust seal. In an alternative embodiment, a septum contained within the first fluid cylinder 310 (and second fluid cylinder 320, if included) could be used instead of a seal that is punctured by the piercing elements.

Optional cylinder extension 330 can extend from the first fluid cylinder 310 toward the proximal end 204. In use, as will be described below, a portion of the priming cap 414, when the priming mechanism 400 is primed (FIG. 4), presses the cylinder extension 330 to displace the cylinder retainer 240 and actuate the first fluid cylinder 310. In this embodiment, following priming the first piercing element 352 punctures the first seal 314 (and second piercing element 354 with the second seal 324, if included).

Optional plunger engagement tab 340 (FIG. 2), extends from the fluid holding system. This plunger engagement tab 340 slidably receives into a tab channel 521 of the actuation control mechanism 500 to securely, slidably connect the fluid holding system 300 with the actuation control mechanism 500, and in particular with the plunger 520.

In some embodiment, its advantageous to include both the first liquid 311 and second liquid 321. For example, the first liquid 311 and second liquid 321 could be materials that during storage are incompatible and separated from one another but have advantages to be used together. For example, the first liquid 311 and second liquid 321 could be reactive with one another and therefore must be separated during storage, and in use upon mixing a change occurs. In one embodiment, the first liquid 311 and second liquid 321 are a two-part adhesive system, that crosslink together. One example of a two-part adhesive comprises epoxy systems, and one specific example is a hydrophobic oxalate ester and a polyethyleneimine. Each liquid 311 is viscous, and upon contact with the other, the liquids will mix, crosslink, or cure and begin to solidify.

Commonly, adhesives used on skin are a single part cyanoacrylate adhesive that has very low viscosity, exothermic upon curing, and brittle when cured. A dispenser that can control the delivery of a two-part system expands the options for adhesives used for skin applications that are biocompatible, strong, and quick curing.

### Fluid Delivery System

As best seen in FIGS. 2, 3, 4, extending from the fluid holding system 300 is the fluid delivery system 350. The fluid delivery system 350 has a first piercing element 352 and a nozzle 360 at the distal end 202. The first fluid cylinder 310 hold the first liquid 311. The nozzle 360 extends from the first fluid cylinder 310 to dispense the first liquid 311.

The nozzle 360 could dispense the liquid 311, without a separate applicator tip 220. As shown in this embodiment, the nozzle 360 is within the applicator tip 220 such that the applicator tip 220 can be shaped to spread the first liquid 311 in a length or width direction beyond just the nozzle 360. Also, the applicator tip 220 can have a wide edge to allow for scrapping, spreading, or wiping the dispensed first liquid 311.

Various embodiments of applicator tips 220 are shown in FIGS. 9A-D. The applicator tip 220 allows the user to sculpt the liquid 311 during dispensing. The applicator tip 220 can contains a pocket 222 proximal to the location of the nozzle 360. This is shown in FIG. 9D. Actuation of the applicator tip 220 allows the pocket 222 to fill with liquid 311 and releases to control flow rate to the targeted surface independent of the speed of dispensing from the nozzle 360. The curved aspect of the applicator tip 220 as best seen in FIG. 9B controls the dispensed liquid 311 width and height with the tilting of the angle that the dispenser 100 is held against the targeted surface.

The applicator tip 220 could be either soft or rigid depending upon the viscosity of the liquid 311 being dispensed. In addition, the applicator tip 220 may be used to resculpt liquid 311 that has not yet fully set.

In embodiment where there is a second fluid cylinder 320, a separate nozzle could extend from the second fluid cylinder 320 to dispense the second liquid 321. As shown in this embodiment, the nozzle 340 secures to both the first fluid cylinder 310 and the second fluid cylinder 320. In this embodiment, adjacent to the first fluid cylinder 310 and second cylinder 320 is a mixing chamber 356 where the first liquid 311 enters and the second liquid 321 enters before passing to the nozzle 360. A first and second cylinder gaskets 316, 326 can be included in the interface between the nozzle 360 (or mixing chamber 356, if included) and the first fluid cylinder 310, second fluid cylinder 320, respectively. Typically, these cylinder gaskets 316, 326 are flexible, compliant, and rubbery materials. The cylinder gaskets 316, 326, as best seen in FIG. 3, include a recessed portion, and as seen in FIG. 4 a portion of the cylinder gasket 316, 326 enters into the first fluid cylinder 310 and second fluid cylinder 320, respectively, to create a tight seal and prevent leaking of the liquid 311, 321. A portion of the cylinder gasket 316, 326 remains outside of the first fluid cylinder 310 and second fluid cylinder 320 (FIG. 4). Optionally, within the nozzle 430 is a static mixer 364 for enabling mixing of the first liquid 311 and second liquid 321 prior to dispensing form the nozzle 360.

Typically, during storage and prior to use, the first liquid 311 is sealed in the first fluid cylinder 310 by a first seal 314, and the second liquid 321 is sealed within the second fluid cylinder 320 by a second seal 324. Actuation of the fluid holding system 300 from activating the priming mechanism 500 causes the first fluid cylinder 310 to engage with a first piercing element 352 to puncture the first seal 314. Similarly, if included, actuation of the priming mechanism 500 and the fluid holding system 300 can cause the second fluid cylinder 320 to engage with a second piercing element 354 to puncture the second seal 324. The seal 314, 324 is typically a puncturable film and may be a multilayer laminate that is welded or otherwise secured to the cylinders to for a robust seal. In an alternative embodiment, a septum contained within the first fluid cylinder 310 (and second fluid cylinder 320, if included) could be used instead of a seal that is punctured by the piercing elements.

The piercing element 352, 354 is shaped with a tip 353, 355 to pierce, puncture, slit the seal 314, 324. The remaining portion of the piercing element 352, 354 is wider and larger than the tip 353, 355 to press the pierced, punctured, slit seal 314, 324 away from the fluid path withing the piercing element. Therefore, the punctured seal 314, 324 is less likely to block the dispensed liquid 311, 321. FIG. 10A-FIG. 10F show alternative embodiments of a piercing element 352, 354 with tip 353, 355.

### Priming Mechanism

The priming mechanism 400 has a priming button 410, a spring 420, and at least one, and here several priming latches 430. Optionally, within the priming button 410 there is a spring seat 412 for securely holding the spring 420. Optionally, directly at the proximal end 204 is a priming cap 414, which could be separable from the priming button 410 and hold the spring seat 412.

The priming mechanism 400 moves relative to the housing 200 to compress or further compress the spring 420. In this embodiment, the priming mechanism 400 is at the proximal end 204 of the housing 200. In a first unprimed position, the spring 420 could be in a rest position or slightly compressed. In a second primed position, where the priming mechanism 400 has moved towards the distal end 202, the spring 420 becomes more compressed. Also, the priming latch 430 secures within a priming active retainer 232 to hold the priming mechanism 400 and the compress spring 420 in position.

### Actuation Control Mechanism

The actuation control mechanism 500 includes an actuation button 510 and a slidable plunger 520. The actuation control mechanism 500 controls the start and stop of the dispensing of the contained liquid 311, 321. FIG. 7 shows a perspective, sectional view of a portion of the actuation button 510 and slidable plunger in a locked engagement position.

The actuation button 510 is accessible from the external portion of the housing 200. The actuation button 510 can be pressed inward into the housing 200. In this embodiment, the actuation button 510 has connects with the housing at a hinge 514 while separated from the housing at a frame 515 in the remaining portions of the actuation button 510 apart from the hinge 514. This arrangement allows for the actuation button 510 to flex at the hinge 514 and because disconnected at the frame 515, the actuation button 510 can press inward into the housing 200. Other embodiments of an actuation button 510 could be included so long as it inwardly flexes relative to the housing 200.

Connected with the actuation button 510 are actuation engagement extensions 512, each separated from the adjacent by an actuation extension gap 513. As can be seen in this embodiment, these actuation engagement extensions 512 are similar to a series of teeth or gears. Because the actuation engagement extensions 512 are connected with the actuation button 510, movement of the actuation button 510 causes similar movement of the actuation engagement extension 512.

The plunger 520 provides the contact connection with the priming mechanism 400 and the selective engagement and disengagement with the actuation control button 510 to initiate or stop dispensing the liquid 311, 321 from the fluid holding system 300. The plunger 520 includes a displacement arm for each fluid cylinder. In this embodiment, the plunger 520 has a first fluid displacement arm 522 to engage with the first fluid cylinder 310 to press the first liquid 311 from the first fluid cylinder 310. The plunger 520 has a second fluid displacement arm 524 to engage with the second fluid cylinder 320 to press the second liquid 321 from the second fluid cylinder 320.

A spring abutment surface 530 of the plunger 520 is adjacent to the spring 420. Pressing force from the spring 420, in a first unprimed or second primed position, presses on the spring abutment surface 530.

The plunger 520 includes plunger engagement extensions 526, each separated from the adjacent by a plunger extension gap 528. As can be seen in this embodiment, these plunger engagement extensions 526 are similar to a series of teeth or gears. At a time of locked engagement, (i) the plunger engagement extensions 526 fit with the actuation extension gaps 513 and (ii) the actuation engagement extensions 512 fit within the plunger extension gaps 528. To disengage, the actuation button 510 is pressed, which causes the connected actuation engagement extensions 512 and actuation extension gaps 513 to also move and disconnect from engagement with the plunger engagement extensions 526 and plunger extension gaps 528. When the spring 420 is pressing on the spring abutment surface 530, and the actuation control mechanism is disengaged, then the plunger 520 moves longitudinally towards the distal end 202, thereby engaging with the fluid holding system 300. The plunger 520 strikes the pistons 312, 322 with a momentum from a gap between the plunger 520 and the pistons 312, 322. This momentum acts to release the piston 312, 322 from the cylinder internal wall 310, 320. The pistons 312, 322 are then driven through the cylinder. As best seen in FIG. 2, the end of the pistons 312, 322 at the distal end 202 are slightly flared to expand against the cylinder 310, 320 inner wall as the piston traverses during dispensing (typically the cylinder is drafted, to be narrower at the piston end and wider at the seal end) so that there isn't increasing friction during dispensing.

### Use

FIGS. 3-6 show the various steps of using the dispenser 100. Not all reference numbers are used in these figures to show the actuation.

FIG. 3 is a section view of the assembled dispenser 100 of FIG. 1 in a stored, un-primed position. In the stored, unprimed position, the liquid 311, 321 is closed and sealed within the respective cylinder 310, 320 to prevent leaking. In this unprimed, the priming mechanism 400 is in the most extended position towards the proximal end 204. The priming latch 430 is in the priming rest retainer 230, which prevents the priming mechanism 400 from dislodging entirely from the housing 200. The spring 420 could be relaxed, but more typically to prevent slipping and rattling, the spring 420 is slightly compressed against the spring abutment surface 530. The engagement of the actuation engagement extensions 512 of the actuation control mechanism 500 with the plunger engagement extensions 526 of the plunger 520 hold the plunger 520 from movement. Also, the cylinder retainer 240 is adjacent to the fluid holding system 300 at a side opposite from the pressure applied from spring 420 to the spring abutment surface 530, and the cylinder retainer 240 provides slight resistance to position the seal 314, 324 from contacting the piercing elements 352, 354 prematurely before priming.

FIG. 4 is a section view of the assembled dispenser 100 of FIG. 1 in a primed position and with the actuation control mechanism 500 in the locked engaged position with the plunger 520. In the primed position, the priming mechanism 400 is longitudinally actuated within the housing 200 towards the distal end 202. The priming latch 430 is in the priming active retainer 232, which holds the priming mechanism 400 in the primed position and prevents the priming mechanism 400 from returning to the unprimed position. For a single use dispenser, this is essentially irreversible and once the device is primed it remains primed. In some embodiment, the dispenser could be reusable, and therefore the priming latch 430 would disengage from the priming active retainer 232. In the primed position, the spring 420 is compressed more than in the unprimed position, as shown in FIG. 3. The compressed spring in the primed position creates the force needed to dispense the fluids.

In the primed position in FIG. 4, when the priming button is moved from a first unprimed (FIG. 3) position to primed position (FIG. 4), the priming mechanism 400 to translate the first cylinder 310 into contact with the first piercing element 352 to create an open fluid path between the fluid holding system 300 and the fluid delivery system 350. A portion (412) of the priming mechanism 400 actuates against the cylinder extension 300 to translate cylinder toward the distal end and break/bend the cylinder retainer 240. This engages the cylinders 310, 320 with the piercing elements 352, 354 to puncture the seal 314, 324. Although in this primed position shown in FIG. 4 the fluid cylinders 310, 320 are engaged with the piercing elements 352, 354 to open the sealed cylinders, the actuation control mechanism 500 still is in a locked engaged position and the plunger 520 has not yet initiated pressing against the cylinders 310, 320 to force dispensing of liquid 311, 321.

FIG. 5 is a section view of the assembled dispenser 100 of FIG. 1 in a primed position and with the actuation control mechanism 500 partially released and dispensing liquid 311, 321. The priming mechanism 400 in in the same position as described in FIG. 4. The actuation control button 510 and the connected actuation engagement extension 512 have been pressed and flexed at the hinge 514 inward toward the housing 200. This causes the actuation engagement extensions 512 to disengage from the plunger engagement extension 526. Therefore, the plunger 520 becomes free to slide relative to the housing 200. The spring 420 presses on the spring abutment surface 530, translating movement of the plunger towards the distal end 202. The displacement arms 522, 524 engage with the first fluid cylinder 310 to force the first liquid 311 to the nozzle 360, and if included engage with the second fluid cylinder 320 to force the second liquid 321 to the nozzle 360. The liquid 311, 321 enters the nozzle 360 and dispenses from the dispenser 100.

If included, the liquid 311, 321 enters a mixing chamber before entering the nozzle 360. If included the liquid 311, 321 passes through a static mixer 364 though the nozzle 360 to achieve mixing of the first liquid 311 with the second liquid 321, prior to dispensing from the dispenser 100. The spring constant effects the rate of dispensing the liquid 311, 321. Therefore, to adjusts for too fast or too slow of dispensing springs of different constants can be utilized.

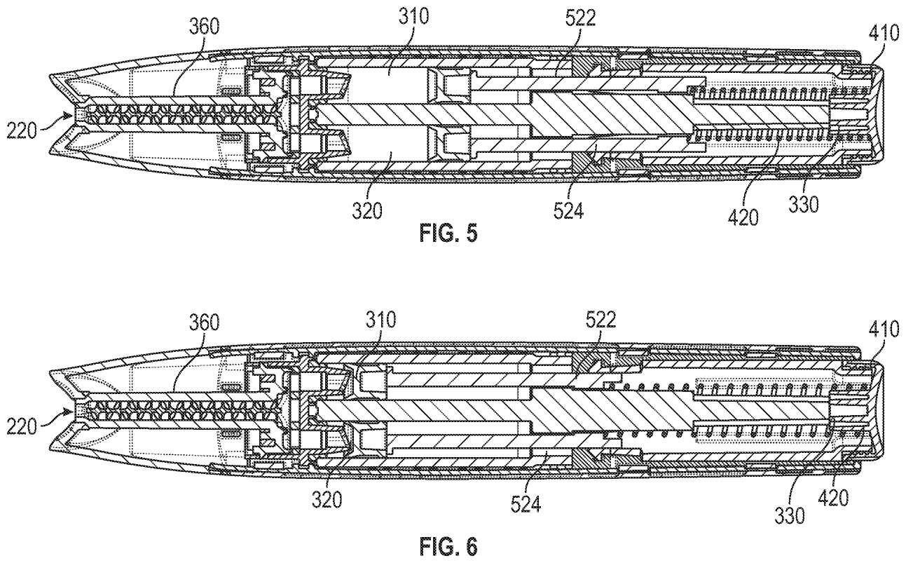

FIG. 6 is a section view of the assembled dispenser of FIG. 1 in a primed position and with the actuation control mechanism 500 fully released and having fully dispensed the liquid 311, 321. The priming mechanism 400 in in the same position as described in FIG. 4. This embodiment functions as described with respect to FIG. 5. Here, the spring 420 has pressed the spring abutment surface 530 and therefore the plunger 520 fully into the fluid cylinder 310, 320.

FIG. 8 is a section view of the assembled dispenser 100 of FIG. 4 showing the path of a first liquid 311 passing from the first fluid cylinder 310 and a second liquid 321 passing from the second fluid cylinder 320 into a mixing chamber 356 and then into the nozzle 360 that contains a static mixer 364.

### Manufacture

The dispenser is manufactured from materials and processes commonly used by those skilled in the art. For example, the dispenser typically is formed from all or a combination of polymeric material, adhesives, metals. For example, rigid materials for parts like the housing, priming mechanism, plungers, and mixing chamber can be formed from polymethylmethacryalate (PMMA), acrylonitrile butadiene styrene (ABS), polycarbonate (PC), or a blend of ABS/PC. Elastomers (for seals and gaskets) or semirigid materials for parts like the pistons can be formed from thermoplastic elastomers, thermoplastic polyurethanes, thermoplastic vulcanizates, silicones, polyethylene (HDPE, LLDPE, MDPE) or polypropylene. The seals can be formed of foils like aluminum ideally with a polymeric layer that is compatible with the surface to which it will bond. The dispenser is manufactured by assembly of individual molded, 3-D printed parts combined, secured, sealed together in commonly used methods. When used as a dispenser of medical goods, the dispenser should be able to withstand sterilization processing.

Although specific embodiments have been shown and described herein, it is understood that these embodiments are merely illustrative of the many possible specific arrangements that can be devised in application of the principles of the invention. Numerous and varied other arrangements can be devised in accordance with these principles by those of skill in the art without departing from the spirit and scope of the invention. The scope of the present invention should not be limited to the structures described in this application, but only by the structures described by the language of the claims and the equivalents of those structures.

1. A liquid dispenser with a proximal end and a distal end, the liquid dispenser comprises:
a housing supporting a fluid holding system, a fluid delivery system, a priming mechanism, and an actuation control mechanism with an applicator tip at the distal end;
the fluid holding system comprising:
a first cylinder with a seal for holding a first liquid;
a first piston slidably within the first cylinder;
the fluid delivery system comprising;
a first piercing element;
a nozzle extending from the first cylinder to the applicator tip;
the priming mechanism comprising;
a priming button slidably connected to the housing;
a spring seated within the priming button;
a priming latch extending from the priming button;
the actuation control mechanism comprising;
a plunger slidably connected to the housing comprising:
a first fluid dispensing arm engaging with the first piston;
a plurality of plunger engagement extensions, wherein each plunger engagement extension is spaced from an adjacent plunger engagement extension;
a spring abutment surface contacting the spring;
an actuation button connected with a plurality of actuation engagement extensions, wherein each actuation extension is spaced from the adjacent actuation extension by an actuation extension gap;
wherein the priming button is movable from a first unprimed position to a second primed position to translate the first cylinder into contact with the first piercing element and creating an open fluid path between the fluid holding system and the fluid delivery system.

2. The liquid dispenser of claim 1, wherein in the second primed position the priming latch locks with a priming active retainer in the housing to restrict movement in a proximal direction and to compress the spring against the spring abutment surface of the plunger.

3. The liquid dispenser of claim 1, wherein in the second primed position the spring is compressed more than in the first unprimed position and the spring in the primed position creates force for dispensing of the first liquid.

4. The liquid dispenser of claim 1, wherein the actuation button is movable from a first engaged position with the actuation engagement extensions interconnected with the plunger engagement extensions to restrict movement of the plunger to a second disengaged position with the actuation engagement extension disengaged from the plunger engagement extensions.

5. The liquid dispenser of claim 1,
wherein the fluid holding system further comprises:
a second cylinder holding a second liquid; and
a second piston slidably within the second cylinder;
wherein the plunger further comprises a second fluid dispensing arm engaging with the second piston;
wherein the fluid delivery system further comprise a second piercing element.

6. The liquid dispenser of claim 5, wherein the nozzle receives the first liquid and second liquid.

7. The liquid dispenser of claim 1, wherein the nozzle comprises a static mixer.

8. The liquid dispenser of claim 1, further comprising a mixing chamber connecting the first cylinder and second cylinder to the nozzle.

9. The liquid dispenser of claim 1, wherein the applicator tip comprises a recessed pocket adjacent the nozzle.

10. The liquid dispenser of claim 1, further comprising a cylinder gasket surrounding the first piercing element to form sealing contact between the housing and the pierced first cylinder.

11. The liquid dispenser of claim 1, wherein the first piercing element comprises a tip and body, wherein the body aligns substantially within the first cylinder to displace the seal.

12. The liquid dispenser of claim 1, wherein the actuation button is accessible at the housing.

13. The liquid dispenser of claim 1, wherein the actuation button is in the first engaged position in a resting position, and displacement of the actuation button into the housing moves to a second disengaged position.

14. The liquid dispenser of claim 1, where the priming button in the first unprimed position the priming latch locks with a priming rest retainer in the housing to restrict movement in a proximal direction. Page 5 of 6

15. The liquid dispenser of claim 1, comprising a plurality of priming latches.

16. The liquid dispenser of claim 1, wherein the fluid holding system further comprises a plunger engagement tab that slidably is received within a tab channel in the plunger.

17. A method of dispensing liquid from the dispenser of claim 1, comprising:
moving the priming mechanism to the second primed position, wherein the priming latch is retained in a priming active retainer and the spring is compressed against the spring abutment surface;
actuating the actuation button to disengage the actuation engagement extensions from the plunger engagement extensions such that the spring slidably moves the spring abutment surface of the plunger and a first fluid displacement arm in the first fluid cylinder.

18. The method of claim 17, further comprising:
actuating the actuation button to engage the actuation engagement extensions the plunger engagement extensions to stop slidable movement of the plunger.
